(19) United States
(12) Patent Application Publication
(10) Pub. No.: US 2012/0115801 A1
(43) Pub. Date: May 10, 2012

(54) METHOD FOR OBTAINING A STABLE DISPERSION OF BENZOYL PEROXIDE

(75) Inventor: Gordon Jay Dow, Santa Rosa, CA (US)

(73) Assignee: Dow Pharmaceutical Sciences, Inc., Petalmua, CA (US)

(21) Appl. No.: 13/352,712

(22) Filed: Jan. 18, 2012

Related U.S. Application Data
(62) Division of application No. 12/589,236, filed on Oct. 20, 2009.
(60) Provisional application No. 61/196,669, filed on Oct. 20, 2008.

(51) Int. Cl. A61K 31/327; A61P 17/10; A61P 31/04; A61P 17/00; A61K 31/7056; A61K 31/7052

(52) U.S. Cl. 514/28; 514/714; 514/23

(57) ABSTRACT

Powder containing benzoyl peroxide is readily wetted by contacting the powder with a liquid containing one or more of a polyol, a polyol, ether, and a low-carbon organic alcohol.

This application is a divisional of pending U.S. patent application Ser. No. 12/589,236, which was filed on Oct. 13, 2009, which claims priority from U.S. Provisional Patent Application Ser. No. 61/196,669, which was filed on Oct. 20, 2008 and which is incorporated herein by reference.

### FIELD OF THE INVENTION

The invention pertains to the field of formulating stable dispersions and microdispersions of benzoyl peroxide.

### BACKGROUND OF THE INVENTION

Benzoyl peroxide is used extensively in dermatologic pharmaceutical compositions. Many compositions for the treatment of acne vulgaris and acne rosacea, for example, contain between 2.5% and 10% benzoyl peroxide. The effectiveness of benzoyl peroxide in treating these and other dermatologic conditions is its usefulness as a keratolytic agent, thereby increasing skin turnover and clearing pores. Benzoyl peroxide additionally has direct antibacterial activity as well.

A serious difficulty in obtaining stable dispersions of benzoyl peroxide in aqueous fluids is that benzoyl peroxide is a highly hydrophobic organic compound and is not readily wetted by water. This problem has been dealt with by the prior art in one or more ways.

Benzoyl peroxide may be dissolved in an organic solvent, thus avoiding the problem of preparing a homogeneous, cosmetically elegant and efficacious dispersion of benzoyl peroxide for topical administration for treating a skin affliction. Early products containing benzoyl peroxide in solution for topical use were gels in which the benzoyl peroxide was dissolved in an organic solvent such as acetone or a combination of alcohol and acetone. These products proved to be efficacious, however they suffered from several disadvantages including flammability, over-drying the skin, and causing frank skin irritation in many acne sufferers. More recent developments have used other organic solvents to solubilize benzoyl peroxide. However these compositions do not solve the problem of severe skin irritation in a significant number of subjects due to the problem of bolus delivery of solubilized benzoyl peroxide into the pilo-sebaceous apparatus of the skin.

For these and other reasons, including increased production of degradation products that occurs with solutions, suspensions of benzoyl peroxide are preferred over solutions. Micro-suspensions, that is suspensions containing micronized benzoyl peroxide, are preferred to standard or non-micronized suspensions of benzoyl peroxide for several reasons, including the following exemplary reasons. First, micronized suspensions provide effective delivery of small particles of benzoyl peroxide into the infundibulum of the pilo-sebaceous apparatus, in which they lodge and from which they provide non-bolus delivery of drug into the sebum and pilo-sebaceous tissue. This delivery provides a proper balance of optimal efficacy and reduction of skin irritation reactions. Second, cosmetic elegance and patient acceptance is improved with the smooth, homogeneous gels, creams or lotions containing suspended micronized benzoyl peroxide. Particularly in treating facial conditions of the skin such as acne or acne rosacea, cosmetic elegance is an important factor in obtaining good patient compliance with treatment instructions. For chronic diseases with ongoing topical drug management, good patient compliance is essential in obtaining overall treatment success.

Surfactants are often utilized as wetting agents to help disperse benzoyl peroxide in aqueous fluids and to maintain benzoyl peroxide in stable suspension. Surfactants, however, are often irritating to damaged or diseased skin and, when applied to intact skin repeatedly, surfactants are known to disrupt the normal skin barrier function as evidenced by an increase in trans-epidermal water loss from the skin. Therefore it is desirable to formulate pharmaceutical compositions, particularly those that will be used daily over extended periods for treating chronic skin conditions, with minimal or no surfactants. In order to facilitate dispersion of benzoyl peroxide and to maintain the dispersion of benzoyl peroxide in suspension, a micronized form of benzoyl peroxide is often utilized, sometimes in conjunction with a surfactant.

Cox, U.S. Pat. No. 3,535,422, discloses a stable emulsion containing benzoyl peroxide. Cox discloses two methods to obtain the emulsion containing benzoyl peroxide in suspension. In a first method, Cox forms an emulsion containing water, a surfactant, and up to 25% of a saturated organic compound emollient. Dry micronized benzoyl peroxide is then blended into this emulsion to obtain the composition. In a second method, utilizing non-micronized benzoyl peroxide, coarse crystals of benzoyl peroxide in the form of a powder packaged wet with water are combined with a previously made emulsion containing all of the components of the composition, including a surfactant and a saturated organic compound emollient. The resulting composition is then milled in order to obtain a composition containing micronized benzoyl peroxide.

Young, U.S. Pat. No. 4,056,611, discloses a single-phase composition containing benzoyl peroxide in suspension. The composition of Young contains an alcoholic solvent, water, and a surfactant as necessary components. Like Cox, Young discloses that the composition may be made by using dry micronized benzoyl peroxide crystals. Preferably, Young utilizes, as does Cox, a wet-packed powder of coarse crystals of benzoyl peroxide, which powder contains 70% benzoyl peroxide and 30% water w/w. All of the components of the composition are mixed together and then this mixture is milled to obtain a composition containing micronized benzoyl peroxide in suspension. Young further discloses that the compositions may advantageously contain a suspending agent to maintain the benzoyl peroxide particles in suspension and a viscosity building (gelling) agent.

The Cox and Young methods and compositions contain several disadvantages pertaining to compositions containing benzoyl peroxide. In both Cox and Young, surfactants are utilized, which are often irritating to damaged or diseased skin. Further, both Cox and Young disclose combining together all constituents of their compositions containing coarse, non-micronized benzoyl peroxide to form a mixture and then milling this mixture to obtain a composition containing micronized benzoyl peroxide. Although Young discloses that a gelling agent may be combined in the composition, it is well known that the mechanical milling forces used in to micronize benzoyl peroxide will likewise tend to disrupt the polymers utilized as gelling agents. Thus, the milling process results in a reduction of the ability of the gelling agents to provide the viscosity that is desired.

Klein, U.S. Pat. No. 4,387,107, discloses gel compositions containing benzoyl peroxide. Klein avoids the problem of milling a composition containing benzoyl peroxide by using benzoyl peroxide that is pre-micronized prior to combining with the remaining ingredients. In order to make the composition of Klein, water is combined with a gelling agent to make a first mixture. To this mixture is optionally added an alcohol vehicle and other components such as a perfume and other therapeutic agents such as methyl salicylate. Finally, a second mixture containing micronized benzoyl peroxide, a surfactant, and water is added to the first mixture to obtain the composition. Because micronized benzoyl peroxide is used, there is no need to mechanically mill the composition. Thus, the polymeric gelling agents are not disrupted. However, the method of Klein requires the use of pre-micronized benzoyl peroxide and the presence of a surfactant.

The use of micronized benzoyl peroxide, as disclosed in Klein, provides advantages, particularly regarding the formation of semi-solid compositions containing one or more polymeric gelling agents. Micronized, as opposed to non-micronized benzoyl peroxide, is more readily suspended in a hydrophilic fluid and such suspensions are more physically stable than are similar suspensions made with non-micronized benzoyl peroxide. However, micronized benzoyl peroxide, particularly as pharmaceutical grade material, is often difficult to obtain and, when it is obtainable, micronized benzoyl peroxide is expensive. In fact, micronized benzoyl peroxide is not commercially available as a pharmaceutical grade prepared under current Good Manufacturing Practices (cGMP).

It would, therefore, be advantageous to be able to purchase non-micronized benzoyl peroxide, which is readily available in a pharmaceutical grade and is much less expensive than micronized benzoyl peroxide, and to then be able to micronize the benzoyl peroxide for use in manufacturing pharmaceutical formulations.

As disclosed in both the Cox and Young patents, benzoyl peroxide, in solid crystalline form, is stable at room temperature but is flammable and capable of exploding when subjected to temperatures associated with grinding. Consequently, dry milling of benzoyl peroxide is not preferred. Rather, it is preferred to wet-mill benzoyl peroxide in order to obtain benzoyl peroxide in a micronized form. Benzoyl peroxide in the presence of water, which is utilized in the preferred wet milling processes, is much safer to process as the risk of fire and explosion is minimized.

One difficulty encountered in wet-milling benzoyl peroxide, as mentioned above, is that benzoyl peroxide is highly hydrophobic and resists wetting with water. Inadequately or non-wetted benzoyl peroxide can interfere with wet-milling, because the benzoyl peroxide powder has a tendency to “crust,” that is it forms a stable crust with entrapped air as it rises to the surface of the dispersing fluid in large amounts due to the buoyancy imparted by air trapped on the nooks and crannies on the surface of the benzoyl peroxide powder. Further, the strong attractive forces between benzoyl peroxide particles create a problem of aggregation which compromises both the manufacturing process and the quality of the final pharmaceutical formulation. Surfactants have been utilized for this purpose and to maintain a stable-non-agglomerated micro-suspension of benzoyl peroxide, as disclosed in each of Cox, Young, and Klein patents, but surfactants are not preferred due to their tendency to irritate damaged or diseased skin. Therefore, a method in which benzoyl peroxide may be readily wetted, and preferably placed into suspension, in a hydrophilic or aqueous fluid, and preferably without the use of surfactants, would be of great benefit.

### DESCRIPTION OF THE INVENTION

It has been unexpectedly discovered that a benzoyl peroxide powder is readily wetted, and a benzoyl peroxide suspension with minimal or no aggregation may be obtained, by combining the benzoyl peroxide, with or without mechanical agitation, with a wetting fluid, preferably aqueous-based, containing a concentration of one or more organic fluids selected from the group consisting of a polyol, a polyol ether, and a low-carbon organic alcohol, sufficient to wet the benzoyl peroxide powder therewith combined. It has further been discovered that this wetting is obtained without the use of wetting agents, such as a surfactant.

As used herein, the term “benzoyl peroxide powder” means any particulate form of benzoyl peroxide. Examples of such particulate forms include granules, crystals, and amorphous powder,, whether coarse, fine, or ultrafine such as a nanoparticulate powder.

As used herein, the term “powder containing benzoyl peroxide” refers to a powder containing a benzoyl peroxide powder and optionally a particulate form of one or more materials other than benzoyl peroxide. For example, a powder containing benzoyl peroxide may contain particles of benzoyl peroxide and one or more other particles or liquids, wherein the concentration of particles other than benzoyl peroxide in the powder is 50% w/w or less. A powder containing benzoyl peroxide may contain a concentration of benzoyl peroxide between 50% and 100%, for example between 50% and 60%, between 60% and 70%, between 70% and 80%, between 80% and 90%, or between 90% and 100%. An example of a powder containing benzoyl peroxide is Hydrous Benzoyl. Peroxide, USP, which is a granular powder containing about 26% water.

As used herein, the term “non-micronized,” when used in reference to a benzoyl peroxide powder, means a powder in which the average benzoyl peroxide particle is 150 microns or greater in size. Conversely, the term “micronized,” when used in reference to a benzoyl peroxide powder, means a powder in which the average benzoyl peroxide particle is less than 150 microns in size. Preferably, but not necessarily, substantially all of the benzoyl peroxide particles in a non-micronized powder are 250 microns or larger.

As used herein, the term “wetting” refers to the spreading of a fluid over and through a powder so that the particles of the powder are individually and discretely encompassed within the fluid. As is known in the art, a powder is considered to be wetted when almost all, such as 90%, of the particles are encompassed within the fluid. For example, contacting a powder with a suitable wetting fluid results in what is referred to as wetting even though a minority of the particles, typically less than 10% of the particles, do not become wetted.

As used herein, the term “polyol” is synonymous with “polyhydric alcohol” and refers to an alcohol that contains more than one hydroxyl group. Examples of polyols include propylene glycol, hexylene glycol, and sugar alcohols.

As used herein, the term “polyol ether” refers to an alcohol that contains more than one hydroxyl group and an ether group. Examples of polyol ethers include diethylene glycol monoethyl ether (ethoxydiglycol) (Transcutol®, Gattefosse Corporation, Paramus, N.J.), ethers of pentaerythritol, ethers of alkylene glycol, ethers of a fatty alcohol, and ethers of a sugar.

As used herein, the term “low carbon organic alcohol” refers to an alcohol having the formula RCH₂OH, wherein R is either H or is a straight or branched alkyl chain of 1 to 7 carbons, or having a ring structure directly connected to a hydroxyl group or connected to a hydroxyl group by a carbon. Examples of low carbon organic alcohols include alkyl and aryl alcohols such as ethyl alcohol, propyl alcohol, isopropyl alcohol, phenol, and benzyl alcohol.

As used herein, the term “mechanical agitation” refers to the application of kinetic energy to a powder in contact with a liquid in order to facilitate wetting of the powder within the liquid. Examples of mechanical agitation include but are not limited to mixing, stirring, shearing, shaking, or blending. Other examples include sonication, vortexing, and milling.

As used herein, the term “aqueous gel” with regards to a pharmaceutical dosage form for topical application means a single phase semi-solid pharmaceutical dosage form comprising a carrier or carrier system that is gelled with a thickening agent such as a polymer wherein the majority of the carrier or carrier system is water, that is 50% w/w or more.

As used herein, the term “agglomeration” means the strong physical attraction between small solid particles, such that a multiplicity of the particles are aggregated into a single larger mass that appears as a single particle, but which may be broken apart with sufficient energy such as mechanical agitation.

In one embodiment, the invention is a method to obtain a wetted powder containing benzoyl peroxide. According to the method of the invention, the powder is placed in contact with a liquid containing a concentration of a polyol, a polyol ether, or a low-carbon organic alcohol that is sufficient to wet the benzoyl peroxide powder therewith combined. The powder and/or the liquid may be mechanically agitated in order to facilitate, hasten, or complete the process of wetting of the benzoyl peroxide powder, such that it, may be effectively dispersed without significant agglomeration or crusting of the particles.

Non-micronized benzoyl peroxide is available as Hydrous Benzoyl Peroxide, USP, which is sometimes erroneously referred to as “wet” benzoyl peroxide. Hydrous Benzoyl Peroxide may contain between 65.0% and 82.0% benzoyl peroxide and typically contains about 74% benzoyl peroxide and 26% water, in order to reduce flammability and shock sensitivity. The benzoyl peroxide in Hydrous Benzoyl Peroxide is not wetted, as this term is used in the art as described above. Hydrous Benzoyl Peroxide is not a paste and the benzoyl peroxide in Hydrous Benzoyl Peroxide is in a microcrystalline state and behaves as a freely flowing granular powder. There is no chemical interaction between water molecules and the benzoyl peroxide powder and the water does not make the core or the inside of the benzoyl peroxide powder wet. Thus, commercially available “wet” benzoyl peroxide is not wetted.

The benzoyl peroxide in the powder may be micronized or may be non-micronized and, therefore, the description herein pertaining to non-micronized powders will be understood to be applicable also to micronized powders. Micronized benzoyl peroxide powders are often commercially available as a wetted powder containing benzoyl peroxide and water. An example of wetted benzoyl peroxide powders are those marketed under the brand name Benox® (Syrgis Performance Initiators, Inc., Helena, Ark.). Because powders containing micronized benzoyl peroxide are already wetted, such powders are not applicable to the wetting embodiment of the present invention. However, the use of wetted powders containing micronized benzoyl peroxide may be applicable to other embodiments of the invention discussed below.

In accordance with the method of the invention for obtaining a wetted benzoyl peroxide powder, a powder containing benzoyl peroxide is placed in contact with a suitable wetting fluid, which wetting fluid contains a polyol, a polyol ether, or a low-carbon organic alcohol in a concentration sufficient to produce wetting of the benzoyl peroxide powder. The powder and the wetting fluid are permitted to remain in contact with one another for a time sufficient for the benzoyl peroxide to become wetted by the wetting fluid. If desired, or if necessary, the powder and the wetting fluid may be mechanically agitated to facilitate or to hasten or to complete wetting.

A preferred organic fluid is a polyol. Preferred examples of polyols include propylene glycol and hexylene glycol. Another preferred organic fluid is a polyol ether. A preferred example of a polyol ether is ethoxydiglycol. A less preferred organic fluid is an alcohol. Alcohols tend to have a lower flash point than the more preferred organic fluids and, when combined with a flammable or explosion-prone substance such as benzoyl peroxide, should be used in combination with a sufficient amount of a non-flammable or high flash point fluid such as water. Moreover, alcohols, in high concentrations, may cause a stinging sensation on broken skin. However, low-carbon organic alcohols are suitable for the organic fluid of the invention, particularly if stinging is not a concern.

The wetting fluid may contain, in addition to one or more of a polyol, a polyol ether, and/or a low-carbon organic alcohol, additional components such as solvents. Such additional components are preferably liquid at the temperature at which the wetting process is performed and are preferably miscible with the polyol, polyol ether, and/or low carbon organic alcohol utilized. A preferred additional component is water. Water is preferred for several reasons, including retardation of flammability and explosion risk during milling and for its usefulness as a preferred carrier in the ultimate semi-solid pharmaceutical formulation, including gels, creams, suspensions, and lotions. Optionally the wetting fluid may contain dissolved solutes such as additional wetting agents or de-aggregation agents.

It has been surprisingly discovered that a wetting fluid that is a liquid containing a one or more of a polyol, polyol ether, and/or low-carbon organic alcohol or containing a mixture of a polyol, polyol ether, and/or low-carbon organic alcohol and water, wherein the concentration (% w/w) of the polyol, polyol ether, and/or low-carbon organic alcohol, is at or above a required level, is capable of wetting a powder containing benzoyl peroxide. The concentration of the polyol, polyol ether, and/or low-carbon organic alcohol in the wetting fluid will vary depending on factors such as the polyol, polyol ether, or low-carbon organic alcohol used, and on the relative volumes of benzoyl peroxide powder, wetting fluid used, and the type and degree of mechanical agitation used. Generally, the concentration of the polyol, polyol ether, and/or low-carbon organic alcohol in the wetting fluid is between 1% and 100% w/w. Preferably, the concentration is about 5% or higher, more preferably about 10% or higher, and most preferably at least about 15%. The term “about” in the preceding sentence is intended to mean an amount that is rounded to be the amount stated. Thus, about 5% means 4.5% or more, about 10% means 9.5% or higher, and about 15% means 14.5% or higher. The powder and the wetting fluid may be mechanically agitated to facilitate, to hasten, or to complete wetting.

In another embodiment, the invention is a wetted benzoyl peroxide powder that is in combination with a liquid containing one or more of a polyol, a polyol ether, and/or a low-carbon organic alcohol, wherein the concentration of the polyol, polyol ether, and/or low-carbon organic alcohol in the liquid is sufficient to wet the benzoyl peroxide powder.

In another embodiment, the invention is a wetted benzoyl peroxide powder that is in combination with a liquid containing one or more of a polyol, a polyol ether, and/or a low-carbon organic alcohol, wherein the concentration of the polyol, polyol ether, and/or low-carbon organic alcohol in the liquid is sufficient to wet the benzoyl peroxide powder and allow for the efficient mechanical micronization and dispersion of the particles.

In another embodiment, the invention is a wetted benzoyl peroxide powder that is in combination with a liquid containing one or more of a polyol, a polyol ether, and/or a low-carbon organic alcohol, wherein the concentration of the polyol, polyol ether, and/or low-carbon organic alcohol in the liquid is sufficient to wet the benzoyl peroxide powder and thereby reduce and control agglomeration and/or crusting of the benzoyl peroxide particles, whether micronized or not, during the manufacturing process of the topical drug product or component thereof.

In another embodiment, the invention is a method for preparing micronized benzoyl peroxide, such as for use in making a topical pharmaceutical formulation containing benzoyl peroxide as an active ingredient.

In another embodiment, the invention is a suspension of benzoyl peroxide. According to this embodiment, the suspension is a single phase composition containing benzoyl peroxide at a concentration of between 1% and 30% w/w, preferably 10% or less, and most preferably 5% or less. The benzoyl peroxide is suspended in a suspending fluid that contains one or more of a polyol, a polyol ether, and/or a low-carbon organic alcohol. The suspending fluid may contain only polyols, polyol ethers, and/or low-carbon organic alcohols. Alternatively, the suspending fluid may contain one or more vehicle fluids that are other than a polyol, polyol ether, and/or low-carbon organic alcohol. An example of a vehicle fluid that is other than a polyol, a polyol ether, or a low-carbon organic alcohol is water.

It is preferred that the suspending fluid contains only a polyol, polyol ether, or low-carbon organic alcohol. If a vehicle fluid other than a polyol, a polyol ether, or a low-carbon organic alcohol is utilized, such as water, such vehicle fluid should be pharmaceutically acceptable and miscible with the one or more polyols, polyol ethers, and/or low-carbon organic alcohols used. Further, the concentration of the one or more polyols, polyol ethers, and/or low-carbon organic alcohols in the suspending fluid should be that which is sufficient, in the absence of the vehicle fluid that is other than a polyol, polyol ether, or low-carbon organic alcohol, to wet a benzoyl peroxide powder therewith combined.

The benzoyl peroxide in the suspension may be micronized or may be non-micronized. If the benzoyl peroxide is non-micronized, the suspension may be treated by a process by which the benzoyl peroxide in the suspension becomes micronized. Suitable micronization processes include milling, grinding, crushing, cutting, impinging, cavitating, and shearing. Wet-milling is a preferred method of micronization.

Non-micronized benzoyl peroxide, when wetted and suspended in accordance with the method of the invention, has a very low tendency to agglomerate or crust and, therefore, there is little or no problem of benzoyl peroxide particles becoming stuck in the small orifices of a media-mill or Gaulin Mill (Delavan, Wis.). Benzoyl peroxide particles that have been wetted in accordance with the method of the invention and then micronized remain in stable suspension and do not agglomerate or crust to a significant extent prior to being incorporated into a pharmaceutical formulation such as a gel, cream or lotion. The stable micro-suspension obtained according to the invention thus results in good pharmaceutical homogeneity and optimal non-bolus delivery into the skin, particularly the pilo-sebaceous apparatus, thus minimizing irritation potential without compromising efficacy.

In another embodiment, the invention is a method for preparing micronized benzoyl peroxide, such as for use in making a topical pharmaceutical formulation containing benzoyl peroxide as an active ingredient. In accordance with this method, a benzoyl peroxide powder is wetted and in suspension as described above, and the benzoyl peroxide suspension is then subjected to appropriate micronization treatment to obtain a suspension containing micronized benzoyl peroxide.

In another embodiment, the invention is a suspension containing micronized benzoyl peroxide, which benzoyl peroxide has been micronized according to the invention. The micronization process and suspension of benzoyl peroxide of the invention are useful in formulating topical pharmaceutical products containing benzoyl peroxide as an active ingredient, especially topical products that are semi-solid dosage forms. The methods of the invention maintain the dispersed micronized benzoyl peroxide in a stable non-agglomerated and non-crusted state for optimal pharmaceutical acceptability without a “shake well before using” label and for optimal drug delivery.

In another embodiment, the invention is a pharmaceutical formulation containing benzoyl peroxide in suspension in a liquid containing one or more of a polyol, a polyol ether, and/or a low-carbon organic alcohol, wherein the concentration of the one or more polyol, polyol ether, and/or low-carbon organic alcohol is sufficient to wet a powder containing benzoyl peroxide at a concentration of the benzoyl peroxide present in the formulation in the absence of all liquid components of the formulation. Preferably, the benzoyl peroxide is micronized. Preferably, the benzoyl peroxide has been micronized according to the present invention. If desired, the pharmaceutical formulation may contain one or more additional vehicle fluids such as water, as described above. The pharmaceutical formulation may further contain excipients commonly utilized in pharmaceutical formulations, such as humectants, emollients, pH stabilizing agents, preservatives, and anti-oxidants.

The concentration of benzoyl peroxide in the pharmaceutical formulation is preferably between 1% and 10% w/w, with a preferred concentration being between 2% to 5%. If desired, an additional agent that is useful in the treatment of dermatologic disorders such as acne vulgaris or acne rosacea may be included in the formulation. Preferably, the additional anti-acne compound is soluble in the solvent or multiplicity of solvents and so is dissolved in the formulation.

One such preferred anti-acne compound is an antibiotic. Preferred antibiotics include those of the macrolide family of antibiotics such as erythromycin, azithromycin, clarithromycin, tilmicosin, and tylosin, and those of the lincomycin family of antibiotics such as clindamycin and lincomycin. A particularly preferred antibiotic to be used in combination with benzoyl peroxide in the formulation of the invention is clindamycin, such as clindamycin hydrochloride or clindamycin phosphate. Additional topical anti-acne active ingredients that may be contained in the formulation of the invention, either with or without the inclusion of an antibiotic, include salicylic acid, azelaic acid, sulfur, sulfacetamide, resorcinol, alpha-hydroxy acids such as glycolic acid, niacinamide, urea, and retinoids such as tretinoin, adapalene, and tazarotene.

The additional anti-acne compound, if present in the formulation of the invention, is preferably present in a concentration in which there is a demonstrable anti-acne effect in the absence of benzoyl peroxide. For example, if clindamycin is present in the formulation of the invention, the concentration of the clindamycin is preferred to be at least 0.5%, such as 1%. Concentrations of clindamycin lower than 0.5% or higher than 1%, such as 2.5% to 5.0% or higher, may be utilized in the formulation.

It is preferred, although not required, that the formulation be in the form of a gel, preferably an aqueous gel. Accordingly, the formulation of the invention may contain a gelling or thickening agent. Any gelling agent that is water-dispersible, is suitable for use on epithelial tissue such as skin, and forms an aqueous gel of substantially uniform consistency, is suitable for use in the composition of the invention. One preferred gelling agent is hydroxypropylcellulose, such as that sold under the tradename KLUCEL® (Hercules Incorporated, Wilmington, Del., USA). Another preferred gelling agent is hydroxyethylcellulose, such as that sold under the tradename NATROSOL® (Hercules Incorporated). Other suitable gelling agents include carboxyvinyl polymers, also known as carbomers, such as are sold under the tradename CARBOPOL® 934, 940, 941, 980, and 981 (B.F. Goodrich Co., Akron, Ohio, USA), ETD 2020™, and ULTREZ® (Noveon, Inc., Cleveland, Ohio, USA). Additional suitable gelling agents are polyvinyl alcohol, polyethylene oxides, propylene glycol alginates, methylcellulose, hydroxypropylmethylcellulose and natural polymeric gums such as xanthan, and carrageenan. The concentration of gelling agent in the composition may be varied depending on several factors, including the desired viscosity of the gel composition.

If desired, the formulation of the invention may further include additional pharmaceutically acceptable excipients typically used in formulations and known to those skilled in the art. Such excipients include, for example, humectants, emollients, pH stabilizing agents, preservatives, and anti-oxidants.

The semi-solid dosage from of the pharmaceutical formulation of the invention may also be in the form of an emulsion, such as a cream or lotion. Preferably, such creams or lotions are formulated without surfactants due to the tendency of surfactants to be irritating to the skin or to impair the skin barrier function. Thus, it is preferred that the cream or lotion formulations of the invention are made with high molecular weight polymeric emulsifiers which do not exhibit such detrimental effects on skin, such as disclosed in Dow, U.S. Pat. No. 7,368,122, or with low levels of mild emulsifiers such as poloxamers.

The invention is further described in the following non-limiting examples. In the examples that follow, the invention is illustrated primarily with a polyol, specifically propylene glycol, with a polyol ether, specifically ethoxydiglycol, and with a low-carbon organic alcohol, specifically ethyl alcohol. It is understood, however, that the examples are illustrative and that the invention may be practiced with other polyols, other polyol ethers, and/or with other low-carbon organic alcohols.

### EXAMPLE 1

### Wetting of a Benzoyl Peroxide Powder Utilizing a Polyol

A benzoyl peroxide wettability study was conducted as follows. 1.5 grams of a hydrous benzoyl peroxide powder was spread on the surface of each of three test fluids contained in glass beakers having about a 5 cm diameter, containing either 30 ml of purified water (Sample A), 30 ml of a fluid composed of 5% propylene glycol and 95% purified water (Sample B), or 30 ml of a fluid composed of 50% propylene glycol and 50% purified water (Sample C). At the bottom of each beaker was a 12 mm×8 mm magnetic stir bar. Each of the fluids, with the benzoyl peroxide powder on the surface, was stirred at 1200 rpm. After 5 and 10 minutes of stirring, the samples were visually inspected for the degree of wetting of the benzoyl peroxide. Good wetting was evidenced by visual determination of little or no agglomeration and/or crusting. It was determined that the wetting of the benzoyl peroxide in Sample A was poor, with little or no visual evidence of wetting. The wetting of the benzoyl peroxide in Sample B was determined to be good, with visual evidence of wetting of a significant portion of the benzoyl peroxide powder. The wetting of the benzoyl peroxide in Sample C was determined to be very good, with visual evidence of wetting of the majority of the benzoyl peroxide powder.

### EXAMPLE 2

### Wetting Benzoyl Peroxide Powder with Propylene Glycol and Water Fluid to Facilitate the Preparation of a Stable Micronized Suspension to be used in Manufacturing a 6.26% Benzoyl Peroxide Topical Gel

A suspension was prepared containing 33.2% w/w hydrous benzoyl peroxide utilizing a dispersing fluid containing 34.0% w/w propylene glycol and 66% w/w water. Into a 200 gallon stainless steel tank, 175 kg of purified water and 90 kg of propylene glycol were combined. The combination was agitated with a propeller mixer to form a mixture. While mixing, 132 kg of hydrous benzoyl peroxide. (74.5% benzoyl peroxide) powder was added. Mixing continued at 1024 rpm for about 20 minutes to wet and disperse the benzoyl peroxide powder and to obtain a benzoyl peroxide suspension.

Upon visual inspection, the suspension appeared to be smooth and free of lumps, with uniformly wetted benzoyl peroxide. The suspension was passed through a Gaulin Mill for micronization using a wet-milling method. The milling procedure proceeded efficiently and without problems (i.e., there was no mill plugging) and a stable micro-suspension was produced. This suspension was incorporated into the final topical dosage form, a 6.26% benzoyl peroxide gel, with the active benzoyl peroxide drug substance present as a stable micro-suspension without the use of surfactants. The propylene glycol content was diluted upon incorporation of benzoyl peroxide suspension into the final topical dosage form to make the 6.26% benzoyl peroxide gel.

### EXAMPLE 3

### Wetting Benzoyl Peroxide Powder with a Fluid Comprising Propylene Glycol and Water to Facilitate the Preparation of a Stable Micronized Suspension to be used in Manufacturing a 3.13% Benzoyl Peroxide Topical Gel

A suspension was prepared containing 24.8% w/w hydrous benzoyl peroxide utilizing a dispersing fluid containing 9.4% w/w propylene glycol and 90.6% w/w water. Into a stainless steel tank, 36 kg of purified water and 3.75 kg of propylene glycol were combined. The combination was agitated with a propeller mixer to form a mixture. While mixing, 13.12 kg of hydrous benzoyl peroxide (74.5% benzoyl peroxide) was added. Mixing continued at 1450 rpm for about 10 minutes to wet and disperse the benzoyl peroxide powder and to obtain a benzoyl peroxide suspension.

Upon visual inspection, the suspension appeared to be smooth and free of lumps, with uniformly wetted benzoyl peroxide. This suspension was transferred to a Gaulin Mill for micronization using a wet-milling method. The milling procedure proceeded efficiently and without problems (i.e., there was no mill plugging) and a stable micro-suspension was produced. This suspension was set aside for a short time before being incorporated into the final topical dosage form, a 3.13% benzoyl peroxide gel, with the active benzoyl peroxide drug substance present as a stable micro-suspension without the use of surfactants.

### EXAMPLE 4

### Wetting Benzoyl Peroxide Powder with a Fluid Comprising Propylene Glycol and Water to Facilitate the Preparation of Astable Micronized Suspension to be used in Manufacturing a Gel Composition Containing Benzoyl Peroxide and Clindamycin

A suspension was prepared containing 24.7% benzoyl peroxide (hydrous) utilizing a dispersing fluid containing 47.6% w/w propylene glycol and 52.4% w/w water. Into a stainless steel tank, 27.5 kg of purified water and 25 kg of propylene glycol were combined. The combination was agitated with a propeller mixer to form a mixture. While mixing, 17.19 kg of hydrous benzoyl peroxide (74.5% benzoyl peroxide) was added. Mixing continued at 858 rpm for about 1 hour to wet and disperse the benzoyl peroxide powder and to obtain a benzoyl peroxide suspension.

The suspension appeared upon visual observation to be smooth and free of lumps, with uniformly wetted benzoyl peroxide. This suspension was passed through a Gaulin Mill for micronization using a wet-milling method. The milling procedure proceeded efficiently and without problems (i.e., there was no mill plugging) and a stable micro-suspension was produced. This suspension was mixed with a gelling agent and set aside for a short time before being combined with a clindamycin solution to form a final topical dosage form, a 2.5% benzoyl peroxide and 1% clindamycin gel, with the active benzoyl peroxide drug substance present as a stable micro-suspension without the use of surfactants. The propylene glycol content was diluted upon combining the benzoyl peroxide suspension with the clindamycin solution to form the final pharmaceutical product.

### EXAMPLE 5

### Wetting of a Benzoyl Peroxide Powder Utilizing a Low-Carbon Organic Alcohol

The wetting study of Example 1 was repeated utilizing 1.5 grams of benzoyl peroxide powder and 30 ml of a fluid composed of 7.5% ethanol and 92.5% water. The fluid with the benzoyl peroxide powder was agitated as described in Example 1. The wetting of the benzoyl peroxide in the fluid was determined to be very good, with visual evidence of wetting of the majority, about 90%, of the benzoyl peroxide powder.

### EXAMPLE 6

### Wetting of a Benzoyl Peroxide Powder Utilizing a Polyol Ether

The wetting study of Example 1 was repeated utilizing 1.5 grams of benzoyl peroxide powder and 30 ml of a fluid composed of 20% polyethylene glycol (PEG 200) and 80% water. The fluid with the benzoyl peroxide powder was agitated as described in Example 1. The wetting of the benzoyl peroxide in the fluid was determined to be very good, with visual evidence of wetting of the majority, about 90%, of the benzoyl peroxide powder.

The above examples show that a hydrophobic benzoyl peroxide powder is easily wetted in water containing a polyol, a polyol ether, or a low-carbon organic alcohol. The wettability of the benzoyl peroxide powder increases with increased concentrations of a polyol, polyol ether, or low-carbon organic alcohol in water and is further facilitated with mechanical agitation. If desired, the benzoyl peroxide powder that has been wetted according to the method of the invention may be effectively and safely micronized by a wet-milling or other process in order to manufacture pharmaceutical formulations containing micronized benzoyl peroxide.

Various modifications of the above described invention will be evident to those skilled in the art. It is intended that such modifications are included within the scope of the following claims.

1. A method for wetting a benzoyl peroxide powder comprising contacting the powder with a wetting fluid containing an organic fluid selected from the group consisting of a polyol, a polyol ether, and/or a low-carbon organic alcohol, wherein the organic fluid is present in the wetting fluid at a concentration that is sufficient to permit the wetting fluid to wet the benzoyl peroxide powder.

2. The method of claim 1 wherein the benzoyl peroxide in the powder is not micronized.

3. The method of claim 2 wherein the powder is Hydrous Benzoyl Peroxide, USP.

4. The method of claim I wherein the wetting fluid contains a solvent in addition to the organic fluid.

5. The method of claim 4 wherein the solvent is miscible with the organic fluid.

6. The method of claim 5 wherein the solvent is water.

7. The method of claim I wherein the organic fluid is a polyol.

8. The method of claim 7 wherein the polyol is propylene glycol or hexylene glycol.

9. The method of claim 1 wherein the organic fluid is a polyol ether.

10. The method of claim 9 wherein the polyol ether is ethoxydiglycol.

11. The method of claim 1 wherein the organic fluid is a low-carbon organic alcohol.

12. The method of claim 1 which further comprises mechanically agitating the wetting fluid in contact with the powder.

13. The method of claim 1 wherein the wetting fluid is free of surfactants.

14. The method of claim 1 wherein the concentration of the polyol, polyol ether, and/or the low-carbon organic alcohol in the wetting fluid is about 5% w/w or higher.

15. The method of claim 14 wherein the concentration is about 10% or higher.

16. The method of claim of claim 14 wherein the concentration is about 15% or higher.

17. A method for making a suspension of benzoyl peroxide comprising suspending a powder that contains benzoyl peroxide in a suspending fluid that comprises one or more of an organic fluid selected from the group consisting of a polyol, a polyol ether, and a low-carbon organic alcohol, wherein the concentration of the polyol, polyol ether, and low-carbon organic alcohol in the suspending fluid is sufficient, even in the absence of any other vehicle fluid that may be included in the suspending fluid, to completely and uniformly wet the benzoyl peroxide powder.

18. The method of claim 17 wherein the benzoyl peroxide in the powder is not micronized.

19. The method of claim 18 wherein the powder is Hydrous Benzoyl Peroxide, USP.

20. The method of claim 17 wherein the suspending fluid contains a solvent in addition to the polyol, polyol ether, and/or low-carbon organic alcohol.

21. The method of claim 20 wherein the solvent is miscible with the polyol, polyol ether, and/or low-carbon organic alcohol.

22. The method of claim 21 wherein the solvent is water.

23. The method of claim 17 wherein the organic fluid is a polyol.

24. The method of claim 23 wherein the polyol is propylene glycol or hexylene glycol.

25. The method of claim 17 wherein the organic fluid is a polyol ether.

26. The method of claim 25 wherein the polyol ether is ethoxydiglycol.

27. The method of claim 17 wherein the organic fluid is a low-carbon organic alcohol.

28. The method of claim 17 wherein the suspending comprises mechanically agitating the suspending fluid and the powder.

29. The method of claim 17 wherein the suspension is made without the use of a surfactant.

30. The method of claim 17 wherein the concentration of the polyol, polyol ether, and/or the low-carbon organic alcohol in the suspending fluid is about 5% w/w or higher.

31. The method of claim 30 wherein the concentration is about 10% or higher.

32. The method of claim of claim 30 wherein the concentration is about 15% or higher.

33. The method of claim 17 which further comprises subjecting the suspension to a micronization treatment and obtaining a suspension containing micronized benzoyl peroxide.

34. A suspension comprising micronized benzoyl peroxide that is obtained by the method of claim 33.

35. The suspension of claim 34 which contains one or more pharmaceutically acceptable excipients.

36. The suspension of claim 34 wherein the concentration of benzoyl peroxide in the suspension is between 1% and 10% w/w.

37. The suspension of claim 36 wherein the concentration of benzoyl peroxide is between 2% and 5%.

38. The suspension of claim 36 which further contains an additional pharmaceutical agent that is useful in the treatment of a dermatologic disorder.

39. The suspension of claim 38 wherein the dermatologic disorder is acne vulgaris or acne rosacea.

40. The suspension of claim 38 wherein the pharmaceutical agent is an antibiotic.

41. The suspension of claim 40 wherein the antibiotic is clindamycin.

42. The suspension of claim 40 wherein the antibiotic is azithromycin.

43. The suspension of claim 36 which is in the form of a semi-solid.

44. The suspension of claim 43 wherein the semi-solid is selected from the group consisting of a cream, a lotion, and a gel.

45. A method for making micronized benzoyl peroxide comprising obtaining a suspension of benzoyl peroxide wherein the benzoyl peroxide is suspended in a suspending fluid that comprises one or more of an organic fluid selected from the group consisting of a polyol, a polyol ether, and a low-carbon organic alcohol, wherein the concentration of the polyol, polyol ether, and low-carbon organic alcohol in the suspending fluid is sufficient, in the absence of any other vehicle fluid that may be included in the suspending fluid, to completely and uniformly wet the benzoyl peroxide powder, and subjecting the suspension to a micronization treatment to obtain the micronized benzoyl peroxide.

46. The method of claim 45 wherein the suspending fluid is free of surfactant.

47. The method of claim 45 wherein the concentration of benzoyl peroxide in the suspension is between 1% and 10% w/w.

48. The method of claim 45 wherein the concentration of benzoyl peroxide is between 2% and 5% w/w.
